(19) United States
(12) Patent Application Publication
(10) Pub. No.: US 2007/0098746 A1
(43) Pub. Date: May 3, 2007

(54) Multi-layered coating technology for taste masking

(76) Inventors: William Michael Nichols, Somerset, NJ (US); Willy Lee, Bridgewater, NJ (US); Neema Kulkarni, Randolph, NJ (US)

Correspondence Address: Johnson & Johnson, 201 TABOR ROAD, MORRIS PLAINS, NJ, 07950, US

(21) Appl. No.: 11/265,644

(22) Filed: Nov. 2, 2005

(51) Int. Cl. A61K 9/50; A61K 9/00

(52) U.S. Cl. 424/400; 424/490

(57) ABSTRACT

Various embodiments of the present invention relate to compositions and methods that have multi-layer coating technology that provide improved taste masking. This technology enables taste masked drug substances to withstand hydration due to saliva in the mouth and aqueous solutions.

### FIELD OF THE INVENTION

The present invention relates to compositions and methods that have improved taste-masking capability.

### DESCRIPTION OF RELATED ART

Many active ingredients or drug substances have objectionable taste characteristics. Attempts to conceal the unpleasant taste by coating the drug substance have been unsuccessful for a number of reasons. First, the coatings themselves often contain defects that result in the leaking or transfer of the unpleasant-tasting active ingredients to the person taking the drug. Additionally, polymers used to coat or encapsulate an active ingredient may swell in the mouth or during manufacturing, which affects the permeability of the encapsulated particle. In certain cases, the coating can rupture which creates pores enabling the drug to leach out. The swelling can also allow saliva from a person or an aqueous solution from a manufacturing process to enter into the encapsulated particle causing the active ingredients to solubilize or leak resulting in an undesirable taste. Further, in cases where the drug substances are bitter and hydrophilic, a large amount of coating may be required to provide satisfactory taste masking when coated using an aqueous vehicle. Thicker coatings are often associated with grit and larger particles and, as such, are typically unsuitable in orally disintegrating dosage forms. Attempts to mask the taste by choosing solvent vehicles in which the drug is either sparingly or not soluble with the aim of minimizing the drug permeation in the coating are unduly expensive and require solvent recovery systems. These methods may substantially alter the release profile or bioavailability of the drug substance. Thus, it would be desirable to provide compositions and methods that mask the bitter taste of drug substances.

### SUMMARY OF THE INVENTION

In one embodiment of the present invention, there is provided an aqueous suspension that includes water, a drug substance, a first coating on the drug substance; and a second coating on the first coating on the drug substance; wherein at least one of the coatings is insoluble in aqueous solutions having a pH greater than about 6.

Another embodiment of the present invention provides for a composition that includes a drug substance; a first coating on the drug substance, and a second coating on the first coating of the coated drug substance; wherein at least one of the coatings is insoluble in aqueous solutions having a pH greater than about 6 and the composition is an immediate release composition.

Another embodiment provides a film that includes a single layer standalone film that is capable of disintegrating in a buccal cavity, wherein the film includes a coated drug substance with a first coating and a second coating. The composition may be an immediate release composition and at least one of the coatings may be insoluble in aqueous solutions having a pH greater than about 6

Useful compositions are immediate release compositions wherein the coatings do not significantly alter the release of the drug substance or its bioavailability when compared to the uncoated drug product.

In one aspect of the present invention, there is provided a multi-layer encapsulation method that provides improved taste masking and an improved moisture barrier to active ingredients, while maintaining or substantially not altering bioavailability of the active ingredients. In one aspect, the invention provides a product and a process for manufacturing a drug product that does not result in the detection of an unpleasant taste by the person taking the drug product. The product may be in a solid dosage form, such as a fast disintegrating fast melt capsules or tablets, powders, wafers, lozenges, center-filled lozenges, soft chew tablets, films including standalone single layer films and multiple-layer films.

In one aspect of the present invention, there is provided a method for making a consumable product which includes encapsulating a drug substance in a first coating, encapsulating the coated drug substance in a second coating, and thereafter forming the consumable product. The coatings mask the taste of the drug substance and may have the same or dissimilar properties.

In another aspect, an aqueous suspension is provided that includes a coated drug product that includes a drug substance, a first coating encapsulating the drug substance, where the first coating masks the taste of the drug substance, and a second coating encapsulating the coated drug substance, where the second coating masks the taste of the first-coated, drug substance.

In another embodiment, there is provided a composition including a drug substance, a first coating encapsulating the drug substance, where the first coating further masks the taste of the drug substance, and a second coating encapsulating the coated drug substance, where the second coating masks the taste of the first-coated, drug substance.

The first and second coatings may be insoluble and/or may remain substantially unswellable in water solutions having a pH greater than about 6. The coatings may have a glass transition temperature or melting point temperature that is greater than about ambient temperature or, in another aspect, greater than about 20° C. in another aspect, greater than about 25° C. or in another embodiment greater than about 40° C. Additionally, the various coatings may have molecular weights greater than about 5,000, or greater than about 10,000 or combinations thereof.

### DETAILED DESCRIPTION OF THE PREFERRED EMBODIMENTS

The terms “immediate release” and “modified release” or “sustained release” or “controlled release” are used in the instant invention as they are commonly understood in the pharmaceutical industry. For immediate release products in solid dosage form (such solid dosage forms including but not limited to tablets, capsules, powders and films), release is defined as the amount of drug substance measured using an appropriate USP or other dissolution test procedure with distilled water as the medium. If no official USP dissolution test procedure has been established, then the most appropriate USP dissolution test will apply, utilizing distilled water as the medium and taking measurements at appropriate time points up to and including the final dosing interval or an appropriate method for equivalent testing of a drug product. For immediate release products in non-solid dosage form (such non-solid dosage forms including but not limited to liquids, syrups, elixirs, liquid center oral products, creams, pastes and gels), release is defined as the amount of active drug substance measured after mixing a 1% solution of the product in distilled water at 37 C for 30 minutes.

For modified, sustained or controlled release products in solid dosage form (such solid dosage forms including but not limited to tablets, capsules, powders and films), release is defined as the amount of drug substance measured using an appropriate USP dissolution test procedure with distilled water as the medium. If no official USP dissolution test procedure has been established, then the most appropriate USP dissolution test will apply, utilizing distilled water as the medium and taking measurements at appropriate time points up to and including the final dosing interval. For modified, sustained or controlled release products in non-solid dosage form (such non-solid dosage forms including but not limited to liquids, syrups, elixirs, liquid center oral products, creams, pastes and gels), release is defined as the amount of drug substance measured after mixing a 1% solution of the product in distilled water at 37 C for 1 hour and taking at least one additional measurement up to and including the final dosing interval.

The following terms are considered to be equivalent and are used interchangeably within this specification: drug substance, active pharmaceutical ingredient, pharmaceutically active agent and active. Drug product is a product that includes a drug substance.

Various embodiments of the present invention provide a multi-layer coating or encapsulation approach to provide improved taste masking and moisture barrier to active ingredients. In various embodiments, the coating materials are in an amount sufficient to accomplish provide improved taste masking and moisture barrier without altering or significantly altering the release, bioavailability or dosage of the active ingredient. Useful dosage forms include ingestible forms as well as those that disintegrate in the mouth with and without the mechanical action of the teeth. Suitable dosage forms include elixirs, liquids, suspensions, syrups, hard and soft chewable tablets and capsules, fast-melt capsules and tablets, films including single standalone films and multiple layer films, lozenge, center-filled lozenge and seamless capsules.

In certain aspects, useful coatings include those that permit the encapsulated materials to withstand hydration due to saliva in the mouth or contact with aqueous materials during the manufacturing of solid dosage forms. In this manner, the encapsulation approach not only may provide taste masking of bitter hydrophilic drugs but also provide an effective moisture barrier to withstand processing conditions encountered such as when producing immediate-release, quick-dissolving or disintegrating solid dosage forms. This technology also enables taste masked drug substances to withstand conditions inside the buccal cavity where the coated drug substance endures hydration due to saliva in the mouth without substantially affecting the taste masking properties of the drug composition. This is particularly useful for fast disintegrating dosage forms designed to disintegrate in the buccal cavity.

Compositions and methods of forming multiple layered particles and compositions thereof having greater than two layers are contemplated. These coated particles may be integrated into liquid or solid dosage forms.

A first coating layer is beneficial because it may seal the bitter-tasting active ingredient of the drug substance. An organic solvent such as acetone, hexane, methylene chloride, and the like, may be employed during the formation of the first layer to reduce permeation or leaking of the drug into the first coating. The second coating layer may seal any crack or defect that may exist in the first coating layer, which assures the integrity of the taste masking. An organic solvent such as those mentioned above may be employed during the formation of the second layer to reduce permeation or leaking of the drug. The second layer may also seal and provide moisture protection to the single-coated particles, resulting in more robust particles which withstand moisture during manufacture of the coated particle in the solid dosage form or in the mouth of a patient.

In one aspect, the coatings of the invention are selected such that they are insoluble or weakly soluble in water. In another aspect, coatings are employed whereby they either are not swellable or are only slightly swellable, such that no leakage occurs, when in contact with aqueous solutions such as water. In another aspect of the invention, coatings are employed that are soluble in gastric fluid. Particular coating materials may be selected to give varying degrees of solubility or swellability, such that the inner layer may be more or less affected by contact with aqueous or gastric fluids as compared with the outer layer.

Useful coating materials include those having glass transition temperatures (for amorphous polymers) or melting point temperatures (for semi-crystalline polymers) above ambient temperature provide effective coatings. Also useful are materials having glass transition temperatures or melting point temperatures above 40° C. Other effective coatings may be produced where materials are selected on the basis that their glass transition temperatures and melting point temperatures exceed the boiling points of the solvents employed in the process of applying the coating layer.

Various embodiments provide that useful coating materials include, but are not limited to, a material that is substantially insoluble, insoluble, substantially not swellable, not swellable or combination thereof, in water solutions having a pH from about 5 to about 7 or in another embodiment a pH from about 5.5 to about 6.5 or another embodiment a pH in greater than about 6. Several embodiments provide that at least one of the coatings is insoluble in aqueous solutions having a pH greater than about 6. Several embodiments provide that at least one of the coatings remains substantially unswellable in water solutions having a pH greater than about 6.

Useful coating agents include, but are not limited to, cellulose acetate, cellulose acetate phthalate, ethyl cellulose, acrylics, polyvinyl acetate, polyvinyl acetate phthalate, ethylene-vinyl acetate copolymers, polyvinyl butyrate, shellac, wheat protein, zinc wheat protein or zein, gluten and combinations thereof.

In various aspects of the present invention, the coatings may be selected from the group consisting of aminoalkyl methacrylate copolymers, aminoalkyl methacrylate copolymer and magnesium stearate, cellulose acetate and, alternatively, cellulose acetate combined with triethyl citrate and combinations thereof.

Useful coating materials include Eudragits (E100, EPO, RL, RD) sold by Rohm Pharma, Amberlite®, sold by Rohm & Haas, ethyl cellulose, cellulose acetate, hydroxypropyl methyl cellulose, and hydroxypropyl cellulose. Cellulose-based coatings, such as cellulose acetate, may provide improved taste-masking. Other suitable coating polymers include polyvinyl acetate, polyvinyl acetate phthalate, waxes (natural and synthetic), hydrogenated vegetable or animal oils, glycerol mono/di-esters.

In one embodiment, the first coating includes aminoalkyl methacrylate copolymer and in another embodiment the first coating includes aminoalkyl methacrylate copolymer and magnesium stearate. In another embodiment, the second coating includes cellulose acetate.

In another embodiment, at least one of the coatings includes aminoalkyl methacrylate copolymer and another embodiment provides that at least one of the coatings includes aminoalkyl methacrylate copolymer and magnesium stearate and a further embodiment provides that at least one of the coatings includes cellulose acetate.

Lubricants and plasticizers may be added to the coating solutions. Talc may be used as a partitioning agent. Any suitable finely divided hydrophobic material may be employed to replace stearic acid. Stearic acid may act as a lubricant and binding agent and may retard swelling of the coatings.

Another aspect of the invention is directed to non-self adhering film compositions and methods for producing a supple, non-self-adhering film especially suitable for oral delivery such as those described in U.S. Pat. No. 6,596,298. In one embodiment, the film is a standalone single layer film that is capable of adhering to the buccal cavity and disintegrates fast. Fast disintegrating dosage forms include those that disintegrate in the buccal cavity in less than about 60 seconds or less than about 30 seconds or less than about 10 seconds. In various embodiments, the method to make a film includes mixing a film-forming agent and at least one stabilizing agent to provide a film-forming mixture; dissolving water-soluble ingredients, such as the coated particles containing drug substances, in water to provide an aqueous solution; combining the film-forming mixture and the aqueous solution to provide a hydrated polymer gel; mixing oils to form an oil mixture; adding the oil mixture to the hydrated polymer gel and mixing to provide a uniform emulsified gel; casting the uniform gel on a substrate; and drying the cast gel to provide a film. The uniform gel may be a single layer extruded as a film on a paper substrate that is subsequently peeled off the substrate after drying and then cut based on dosing considerations. The film may also be referred to as a flat, foil, paper or wafer type product. Multiple-layer film may be produced by coextrusion or by casting the uniform gel on a previously cast film. As discussed below, the product provides a system for the application and release of a drug and other active substances.

Useful film-forming agents, include but are not limited to, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, wheat, gluten, soy protein isolate, whey protein isolate, casein and combinations thereof. Other useful materials are disclosed in U.S. Pat. No. 6,596,298, the disclosure of which is incorporated by reference in its entirety.

In one aspect, at least one drug substance encapsulated by multiple coatings are combined with a film-forming solution to create an instantaneous-release, film-based medicinal product for the purpose of delivering a drug to a patient while minimizing or eliminating any unpleasant immediate or subsequent taste associated with the product. Under normal dosing conditions, after the product is placed in the mouth of the person receiving the drug, the product disintegrates in the mouth. In one aspect, upon release, the taste-masking substance counteracts the unpleasant taste quality inherent to the drug substance and provides a non-bitter taste immediately post-ingestion of the product. In another aspect, the product retains its taste-masking properties while retaining bioavailability properties of the drug substance when ingested. Of course, chewable tablets, chewable capsules, fast-melt tablets, fast-melt capsules, and other solid dosage forms designed to disintegrate and dissolve in the buccol cavity as well as dosage forms that are designed to disintegrate in the mouth and dissolve in the mouth, stomach or gastro-intestinal tract, where dissolution in the mouth is to be avoided, may be produced according to the principles of the invention. A fast melt product and methods of preparation are described by U.S. Pat. No. 5,576,014, which is incorporated by reference in its entirety.

Various types of manufacturing equipment, including, for example, conventional machines for performing extrusion molding, injection molding, casting, or dip molding, may be employed in any number of configurations to carry out the various processes of the invention. In one embodiment, the devices disclosed in U.S. Pat. No. 6,499,984, the disclosure of which is incorporated by reference in its entirety, provide structures that are useful in practicing the invention. In one embodiment, flow meters and associated control valves are employed to regulate the metered addition of the coated particles encapsulating drug substances and the contents of the film-forming solution; however, any other suitable metering and control devices may be utilized. A twin screw extruder may be utilized as a continuous mixing device, or, more specifically as described in U.S. Pat. No. 6,499,984, a twin screw wet granulator-chopper may be employed; however, any other suitable mixing device may be used.

A wide variety of drug substances may be useful for carrying out the invention, including water-soluble polymers such as those disclosed in U.S. Provisional Patent Application Ser. No. 60/467,339, the disclosure of which is incorporated by reference in its entirety. In addition, the following table provides a list of exemplary drug substances that may be effectively employed according to the invention.

| Pharmaceutically Active Agent | Dose | |
|---|---|---|
| Chlorpheniramine Maleate | 4-12 | mg |
| Brompheniramine Maleate | 4 | mg |
| Dexchlorpheniramine | 2 | mg |
| Dexbropheniramine | 2 | mg |
| Triprolidine Hydrochloride | 2.5 | mg |
| Cetirizine | 5-10 | mg |
| Acrivastine | 8 | mg |
| Azatadine Maleate | 1 | mg |
| Loratadine | 5-10 | mg |
| Phenylephrine Hydrochloride | 5-10 | mg |
| Dextromethorphan Hydrobromide | 10-30 | mg |
| Sildenafil | 25-100 | mg |
| Ketoprofen | 12.5-25 | mg |
| Sumatriptan Succinate | 35-70 | mg |
| Zolmitriptan | 2.5 | mg |
| Loperamide | 2 | mg |
| Famotidine | 5-10 | mg |
| Nicotine | 1-15 | mg |
| Diphenhydramine Hydrochloride | 12.5-25 | mg |
| Pseudoephedrine Hydrochloride | 15-60 | mg |
| Atorvastatin | 5-80 | mg |
| Valdecoxib | 5-20 | mg |
| Amlodipine | 2.5-10 | mg |
| Rofecoxib | 5-25 | mg |
| Setraline hydrochloride | 10-100 | mg |
| Ziprasidone | 20-80 | mg |
| Eletriptan | 10-40 | mg |
| Nitroglycerin | 0.3-0.6 | mg |

Because a wide variety of processes may be utilized and products produced according to the invention, it is understood that the following examples are merely exemplary.

### EXAMPLE 1

To granulated dextromethorphan HBr with 7% hydroxypropylmethyl cellulose (HPMC E5), a first coating layer of 80:20 (w/w) aminoalkyl methacrylate copolymer (Eudragit E100, Degussa):Mg Stearate (Magnesium stearate) and acetone is added to achieve a coating level of 50%. (Coating levels indicate the percentage of coating relative to the finally coated particle (w/w), such that in the above example, a coating level of 50% represents that the coating weight is half of the combined coating and drug weight.) A second coating layer is applied by adding cellulose acetate (CA-398-10NF, Eastman) and acetone to achieve a coating level of 20%. All coatings are performed in a fluidized bed.

### EXAMPLE 2

To granulated dextromethorphan HBr with 7% hydroxypropylmethyl cellulose (HPMC E5), a first coating layer of 80:20 (w/w) aminoalkyl methacrylate copolymer (Eudragit E100, Degussa):Mg Stearate (Magnesium stearate) and acetone is added to achieve a coating level of 40%. A second coating is applied by adding a layer of 95:5 (w/w) ethyl cellulose (Ethocel 10P, Dow):triethyl citrate (TEC, Morflex) and acetone to achieve a coating level of 15%. All coatings are performed in a fluidized bed.

### EXAMPLE 3

To granulated dextromethorphan HBr with 7% hydroxypropylmethyl cellulose (HPMC E5), a first coating layer of 80:20 (w/w) aminoalkyl methacrylate copolymer (Eudragit E100, Degussa):Mg Stearate (Magnesium stearate) and acetone is added to achieve a coating level of 30%. A second coating is applied by adding a layer of 80:20 (w/w) cellulose acetate (CA-398-10NF, Eastman):triethyl citrate (TEC, Morflex) and acetone to achieve a coating level of 10%. All coatings are performed in a fluidized bed.

### EXAMPLE 4

To granulated dextromethorpan HBr with 7% hydroxypropylmethyl cellulose (HPMC E5), a first coating layer of 80:20 (w/w) shellac (Marcoat 125, Emmerson Resources):hydroxypropylmethyl cellulose (HPMC E5) and water is added to achieve a coating level of 40%. A second coating layer is applied by adding cellulose acetate (CA-398-10NF, Eastman) and acetone to achieve a coating level of 10%. All coatings are performed in a fluidized bed.

### EXAMPLE 5

To granulated dextromethorphan HBr with 7% hydroxypropylmethyl cellulose (HPMC E5), a first coating layer of 80:20 (w/w) shellac (Marcoat 125, Emmerson Resources):hydroxypropylmethyl cellulose (HPMC E5) and water is added to achieve a coating level of 30%. A second coating is applied by adding a layer of 95:5 (w/w) ethyl cellulose (Ethocel 10P, Dow):triethyl citrate (TEC, Morflex) and acetone to achieve a coating level of 20%. All coatings are performed in a fluidized bed.

### EXAMPLE 6

To granulated dextromethorphan HBr with 7% hydroxypropylmethyl cellulose (HPMC E5), a first coating layer of 80:20 (w/w) shellac (Marcoat 125, Emmerson Resources):hydroxypropylmethyl cellulose (HPMC E5) and water is added to achieve a coating level of 50%. A second coating is applied by adding a layer of 80:20 (w/w) cellulose acetate (CA-398-10NF, Eastman):triethyl citrate (TEC, Morflex) and acetone to achieve a coating level of 15%. All coatings are performed in a fluidized bed.

### EXAMPLE 7

To granulated dextromethorphan HBr with 7% hydroxypropylmethyl cellulose (HPMC E5), a first coating layer of aminoalkyl methacrylate copolymer (Eudragit E100, Degussa) and acetone is added to achieve a coating level of 30%. A second coating layer is applied by adding cellulose acetate (CA-398-10NF, Eastman) and acetone to achieve a coating level of 15%. All coatings are performed in a fluidized bed.

### EXAMPLE 8

To granulated dextromethorphan HBr with 7% hydroxypropylmethyl cellulose (HPMC E5), a first coating layer of aminoalkyl methacrylate copolymer (Eudragit E 100, Degussa) and acetone is added to achieve a coating level of 50%. A second coating is applied by adding a layer of 95:5 (w/w) ethyl cellulose (Ethocel 10P, Dow):triethyl citrate (TEC, Morflex) and acetone to achieve a coating level of 10%. All coatings are performed in a fluidized bed.

### EXAMPLE 9

To granulated dextromethorphan HBr with 7% hydroxypropylmethyl cellulose (HPMC E5) is added a first coating layer of aminoalkyl methacrylate copolymer (Eudragit E 100, Degussa) and acetone is added to achieve a coating level of 40%. A second coating is applied by adding a layer of 80:20 (w/w) cellulose acetate (CA-398-10NF, Eastman):triethyl citrate (TEC, Morflex) and acetone to achieve a coating level of 20%. All coatings are performed in a fluidized bed.

### EXAMPLE 10

To granulated dextromethorphan HBr with 7% hydroxypropylmethyl cellulose (HPMC E5), a first coating layer of 80:20 (w/w) aminoalkyl methacrylate copolymer (Eudragit E100, Degussa):Mg Stearate (Magnesium stearate) and acetone is added to achieve a coating level of 32%. A second coating may be applied by adding a layer of 80:20 (w/w) cellulose acetate (CA-398-10NF, Eastman):triethyl citrate (TEC, Morflex) and acetone to achieve a coating level of 10%. All coatings are performed in a fluidized bed.

### EXAMPLE 11

Example 11 duplicates previous Example 10 with the exception that a 100 mesh aminoalkyl methacrylate copolymer may be employed instead of a 60 mesh aminoalkyl methacrylate copolymer.

### EXAMPLE 12

To granulated dextromethorphan HBr with 7% hydroxypropylmethyl cellulose (HPMC E5), a first coating layer of aminoalkyl methacrylate copolymer (Eudragit E100, Degussa) and acetone is added to achieve a coating level of 36%. A second coating layer is applied by adding cellulose acetate (CA-398-10NF, Eastman) and acetone to achieve a coating level of 10%. All coatings are performed in a fluidized bed.

The following table presents the testing results of Examples 1-12. The dissolution test is conducted by soaking the coated particles in 0.1N HCl for 45 minutes and measuring the percentage of released dextromethorphan hydrobromide. For the bitterness test, the coated particles are formed into film strips containing 15 mg of dextromethorphan. Each film strip is placed on the tongue of a patient for 1 minute and each patient provides a numerical rating on bitterness from 0 to 10. Dextromethorphan hydrobromide is an extremely bitter and bad tasting drug substance. As shown in the table below, films with two coatings substantially masked the taste of the drug substance, dextromethorphan hydrobromide.

| | Drug Load | Dissolution | Bitterness |
|---|---|---|---|
| Example | (actual %) | Test (%) | Test |
| 1 | 32.5 | 51.14 | 3.8 |
| 2 | 40.3 | 11.4 | 3.9 |
| 3 | 49.5 | 92.8 | 3.8 |
| 4 | 44.9 | 22.3 | 4.2 |
| 5 | 52.7 | 7.0 | 4.8 |
| 6 | 31.2 | 0 | 3.5 |
| 7 | 56.0 | 94.3 | 5.6 |
| 8 | 41.4 | 32.9 | 3.9 |
| 9 | 43.3 | 64.9 | 3.5 |
| 10 | 43.6 | 95.7 | 3.3 |
| 11 | 43.9 | 95.6 | 3.4 |
| 12 | 36.1 | 92.6 | 4.6 |

While the invention has been described in detail and with reference to specific examples thereof, it will be apparent to one skilled in the art that various changes and modifications can be made therein without departing from the spirit and scope thereof.

What is claimed is:

1. An aqueous suspension comprising:
water, a drug substance, a first coating on the drug substance; and a second coating on the first coating on the drug substance; wherein at least one of the coatings is insoluble in aqueous solutions having a pH greater than about 6.

2. The aqueous suspension of claim 1, wherein the first coating is insoluble in water solutions having a pH greater than about 6.

3. The aqueous suspension of claim 1, wherein the second coating is insoluble in water solutions having a pH greater than about 6.

4. The aqueous suspension of claim 1, wherein the first coating remains substantially unswellable in water solutions having a pH greater than about 6.

5. The aqueous suspension of claim 1, wherein the second coating remains substantially unswellable in water solutions having a pH greater than about 6.

6. A composition comprising a drug substance; a first coating on the drug substance, and a second coating on the first coating of the coated drug substance; wherein at least one of the coatings is insoluble in aqueous solutions having a pH greater than about 6 and the composition is an immediate release composition.

7. The composition of claim 6, wherein the first coating is insoluble in water solutions having a pH greater than about 6.

8. The composition of claim 6, wherein the second coating is insoluble in water solutions having a pH greater than about 6.

9. The composition of claim 6, wherein the first coating remains substantially unswellable in water solutions having a pH greater than about 6.

10. The composition of claim 6, wherein the second coating remains substantially unswellable in water solutions having a pH greater than about 6.

11. The composition of claim 6, wherein the first coating has a glass transition temperature or melting point temperature that is greater than about 40° C.

12. The composition of claim 6, wherein the second coating has a glass transition temperature or melting point temperature that is greater than about 40° C.

13. The composition of claim 6, wherein at least one of the coatings includes aminoalkyl methacrylate copolymer.

14. The composition of claim 6, wherein at least one of the coatings includes aminoalkyl methacrylate copolymer and magnesium stearate.

15. The composition of claim 6, wherein at least one of the coatings includes cellulose acetate.

16. The composition of claim 6, wherein said composition is a dosage form selected from the group consisting of elixirs, suspensions, syrups, liquids, hard and soft chewable tablets and capsules, fast-melt capsules and tablets, films including single standalone films and multiple layer films, lozenges including center-filled lozenges and seamless capsules.

17. The composition of claim 6, wherein the coatings do not significantly alter the release of the drug substance.

18. A film comprising a single layer standalone film that is capable of disintegrating in a buccal cavity, wherein said film comprises a coated drug substance with a first coating and a second coating, and the composition is an immediate release composition.

19. The film of claim 18, wherein at least one of the coatings is insoluble in aqueous solutions having a pH greater than about 6.

20. The film of claim 18, wherein at least one of the coatings remains substantially unswellable in water solutions having a pH greater than about 6.
